(19) United States
(12) Patent Application Publication
(10) Pub. No.: US 2025/0241782 A1
(43) Pub. Date: Jul. 31, 2025

(54) Mouth-Guard System

(71) Applicant: Cooper Collins, Magnolia, TX (US)

(72) Inventor: Cooper Collins, Magnolia, TX (US)

(21) Appl. No.: 18/645,228

(22) Filed: Apr. 24, 2024

Related U.S. Application Data
(60) Provisional application No. 63/462,465, filed on Apr. 27, 2023.

(51) Int. Cl. A61F 5/56

(52) U.S. Cl. CPC A61F 5/566

(57) ABSTRACT

A device utilized to assist in stabilizing a user's mouth during sleep and periods of rest to reduce and prevent or reduce grinding, gnashing of teeth, snoring, and sleep apnea. A user can utilize the present invention to assist with reducing the effects of sleep apnea and to assist with reducing the grinding and gnashing of teeth.

### CROSS REFERENCE TO RELATED APPLICATION

This application claims the benefit of U.S. provisional application Ser. No. 63/462,465 filed on Apr. 27, 2023 and incorporates said application by reference.

### TECHNICAL FIELD OF INVENTION

The present invention relates, in general to a device utilized to assist in stabilizing a user's mouth during sleep and periods of rest to reduce and prevent or reduce grinding, gnashing of teeth, snoring, and sleep apnea.

### BACKGROUND OF INVENTION

Many individuals suffer from Bruxism, which is a condition in which an individual often grinds, gnashes or clenches their teeth. Individuals with Bruxism may unconsciously clench their teeth, which can happen while they are awake or asleep (sleep Bruxism). Sleep Bruxism is often thought of as a sleep-related movement disorder and individuals suffering from sleep Bruxism often have other sleep disorders.

Depending on the severity of the Bruxism, different treatment options may exist. Individuals with mild Bruxism may not have to receive any treatment. However, individuals with severe Bruxism may require some type of treatment as the condition can often lead to jaw disorders, headaches, damaged teeth and other problems. Those suffering often require regular dental care for addressing this condition.

While relief is available for individuals experiencing or suffering from Bruxism, the often-prescribed devices are very expensive and not affordable to many patients. Accordingly, a need exists in the art for an affordable system that can help individuals suffering with Bruxism.

Many individuals also suffer from snoring and sleep apnea, which is a condition in which an individual has frequent disruptions while breathing during sleep, which significantly reduce sleep quality and the health of an individual. The most common form of sleep apnea is “obstructive” sleep apnea, which occurs when the muscles and tissues surrounding an individual's airways slacken, constricting or collapsing the airway. Likewise, common snoring issues can disrupt an individual's sleep or even other individuals who sleep nearby the snoring user.

While relief is available for individuals experiencing or suffering from snoring and sleep apnea, the often-prescribed devices are very expensive and not affordable to many patients. Accordingly, a need exists in the art for an affordable system that can help individuals suffering with snoring and sleep apnea.

### BRIEF SUMMARY OF THE INVENTION

The present invention is a mouthguard system that can assist individuals suffering with Bruxism, snoring, and sleep apnea by providing a guard for the teeth to assist in stabilizing a user's teeth, which will prevent a user from grinding their teeth while resting or sleeping—alleviating problems associated with Bruxism—and will prevent a user from having their jaw slacken and constrict their airway—alleviating problems associated with snoring and sleep apnea.

In one embodiment, the device is a mouthguard that is custom fit to a user's mouth, snuggly and securely holding onto a user's teeth. The device is comprised of both an upper and bottom member for protecting the upper and bottom teeth that provide greater comfort and placement to a user. Attached along one or both sides of the upper and bottom members are protruding nodules or attachment members. The nodules may be hourglass-shaped, but the invention is not limited to any particular shape. The midline of the nodules are grooved such that a rubber-band or other affixing mechanism spans between and connects the nodules located on the upper and bottom members, which in turn means the upper and bottom guards are connected and attached to one another such that some movement may be allowed, but movement of the teeth and jaw is restricted in a manner which reduces the grinding, gnashing, and clenching of teeth resulting from Bruxism and prevents the slackening of the jaw and constriction of the user's airway resulting from snoring and sleep apnea. Thus, the device provides greater comfort and stability for the user during their sleep.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 is an illustration of a perspective view of one example of the embodiment of the present invention where the top and bottom members are separated from one another;

FIG. 2A is an illustration of a perspective view of one example of the embodiment of the present invention where the top and bottom members are in contact with one another; and

FIG. 2 is an illustration of an overhead perspective view of one example of the embodiment of the present invention where the top and bottom members are in contact with one another.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising” when used in this specification, specify the presence of stated features, steps, operations, elements, and/or components, but do not preclude the presence of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present disclosure is to be considered as an exemplification of the invention and is not intended to limit the invention to the specific embodiments illustrated by the figures or description below.

Referring now to the drawings, the present invention will be described in more detail.

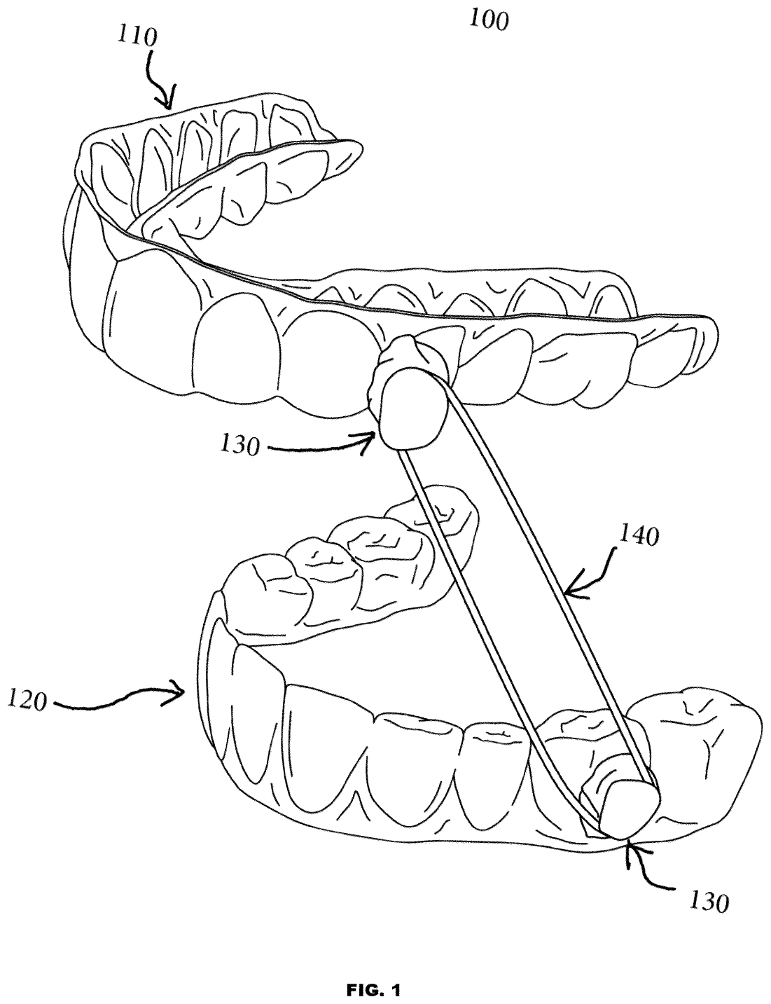

FIG. 1 illustrates a perspective view of one example of the embodiment of the present invention. The mouth guard system 100 in FIG. 1 is illustrated with top member 110 and bottom member 120, separated from one another as they would be if a user had their jaw open. In a preferred embodiment, the present invention is configured so that top member 110 is separate from bottom member 120 and they are two separate pieces. Top member 110 is molded to fit over the top teeth of a user, while bottom member 120 is molded to fit over a user's bottom teeth. Nodule members 130 are affixed to the sides of top member 110 and bottom member 120. While FIG. 1 illustrates nodule members 130 on only one side of mouth guard system 100, the present invention is not limited to this configuration as it may be configured so that nodule members 130 are located on both sides of the mouth guard system 100. And in some embodiments, nodule members 130 may be located at different locations along top member 110 and bottom member 120 of mouth guard system 100 and are not limited to being located on the side of mouth guard system 100.

In one embodiment, as illustrated in FIG. 1, nodule members 130 may be positioned on top member 110 and bottom member 120 of mouth guard system 110 so that one nodule is located in front of the other nodule. For instance, as illustrated in FIG. 1, the nodule member 130 on top member 110 is located towards the front of top member 110 while nodule member 130 located on bottom member 120 is located towards the back of bottom member 120. In other embodiments, the nodule on bottom member 120 may be located towards the front of bottom member 120 while the nodule on top member 110 is located towards the back of top member 110.

In one embodiment, as illustrated in FIG. 1, nodule members 130 may be hourglass-shaped. However, the nodules 130 may be configured in any number of different shapes, such as hook-shaped, square-shaped, and the like. In a preferred embodiment, the midline of the hourglass-shaped nodule members 130 are configured with grooves or notches located at in the middle of nodule member 130 such that an affixing mechanism member 140 spans between and connects the nodules along their midline grooves. The grooves or notches are not limited to being located in the middle of the nodule as the grooves or notches may be located at different locations along the nodule in alternative embodiments. Upper member 110 and bottom member 120 are thus connected to one another by nodule members 130 and affixing mechanism member 140 such that some movement of upper member 110 and bottom member 120 may be allowed, but movement of the user's teeth is restricted in a manner which prevents Bruxism and provides greater comfort and stability for the user.

In one embodiment, the present invention may be configured so that nodules 130 are part of upper member 110 and bottom member 120 or they are separate members that are added to upper member 110 and bottom member 120. In one embodiment, mouth guard system 100 may be configured so that additional nodules 130 may be added to top member 110 and bottom member 120 depending on a user's preference. In one embodiment, the present invention may be configured so that affixing member 140 is a rubber-band or other similar connecting mechanism that loosely connects upper member 110 to bottom member 120. In an alternative embodiment, affixing member 140 may be made of Nylon, Teflon or consist of dental wire, such as orthodontic wires that enable a user to connect upper member 110 to bottom member 120 in either a loose or tight arrangement.

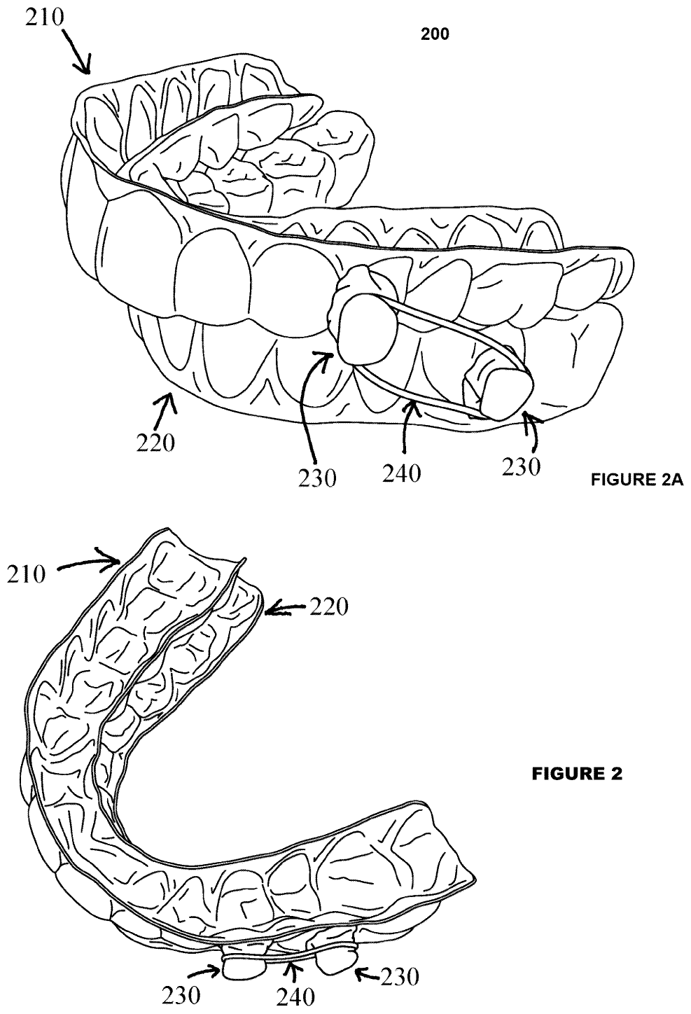

FIG. 2A illustrates a perspective view of one example of the embodiment of the present invention where the top and bottom members are in contact with one another. FIG. 2 is an illustration of an overhead perspective view of one example of the embodiment of the present invention where the top and bottom members are in contact with one another. The mouth guard system 200 in FIG. 2A and FIG. 2 is illustrated with top member 210 and bottom member 220 in contact with one another as they would be if a user had the mouth guard system 200 in their mouth and their jaw closed or clenched. Top member 210 is molded to fit over the top teeth of a user, while bottom member 220 is molded to fit over a user's bottom teeth. Nodule members 230 are affixed to the sides of top member 210 and bottom member 220. The illustrations in FIG. 2A and FIG. 2 are not a limitation on the arrangement or configuration of the present invention as the mouth guard system may be arranged in different configurations.

In one embodiment, as illustrated in FIGS. 2A and 2, nodule members 230 may be hourglass-shaped. However, the nodules 130 and 230 may be configured in any number of different shapes, such as hook-shaped, square-shaped, circular, triangular, or any other number of shapes. In a preferred embodiment, the midline of the hourglass-shaped nodule members 130 and 230 are grooved such that an affixing mechanism member 140 or 240 spans between and connects the nodules along their midline grooves. Upper member 210 and bottom member 220 are thus connected to one another by nodule members 230 and affixing mechanism member 240 such that some movement of upper member 210 and bottom member 220 may be allowed, but movement of the user's teeth and jaw is restricted.

In one embodiment, the mouth guard system 200 is configured so that when affixing member 140 or 240 is utilized it can operate to reduce the grinding, gnashing, and clenching of teeth resulting from Bruxism. By securing the top member 210 and bottom member 220 towards one another with affixing mechanism 240, the system 200 will reduce the grinding, gnashing and clenching of teeth.

In addition, the use of affixing member 240 to secure the top member 210 and bottom member 220 assists in preventing the slackening of the jaw and constriction of the user's airway to assist with snoring and sleep apnea. In one embodiment, nodule members 130 and 230 are adjustable and may be moved along upper members 110 and 210 and bottom members 120 and 220 so that when affixing mechanism 140 or 240 is applied, a user's bottom jaw is pulled forward to assist in opening a user's airway to assist with a user's sleep and help treat sleep apnea. This positioning can be accomplished by adjusting nodules 130 and 230 to a position that is specific to a user to enable the bottom jaw to be pulled forward to assist in opening a user's airway. Thus, the device provides greater comfort and stability for the user during their sleep by preventing slackening of the jaw and reducing constriction of a user's airway.

In one embodiment, the present invention may be configured so that nodules 130 and 230 are sized to accommodate more than one affixing mechanism 140 or 240. The nodules 130 and 230 may be sized to accommodate multiple affixing mechanisms 140 or 240 to help keep the users bottom jaw pulled forward to better assist in opening the user's airway to better help with snoring and sleep apnea and to assist in reducing the grinding, gnashing and clenching of teeth. In one embodiment, nodules 130 and 230 may be sized to accommodate multiple rubber bands to keep upper members 110 and 210 pulled toward bottom members 120 and 220.

FIG. 2 illustrates an overhead perspective view of one example of the embodiment of the present invention where the top member 210 and bottom member 220 are in contact with one another. In addition, FIG. 2 illustrates the operation of nodules 230 with affixing mechanism 240 in place around both nodules with one end of affixing mechanism 240 around the nodule 230 located on top member 210 and the other end of affixing mechanism 240 around the nodule 230 located on bottom member 220. In such arrangement, the combination of the affixing mechanism 240 located around the nodules in combination with top member 210 and bottom member 220 can assist in keeping the users bottom jaw pulled forward to better assist in opening the user's airway to better help with snoring and sleep apnea and also assist in reducing the grinding, gnashing and clenching of teeth and the effects from such grinding, gnashing and clenching of teeth.

While rubber has been mentioned as a possible material for elements of the embodiment described herein, the device and its elements are not limited to any particular materials. In alternative embodiments, members 110, 120, 210, and/or 220 may be made of thermoplastic, polyvinyl chloride, polyurethane, or variations of co-polymers of vinyl acetate or ethylene. The present invention may also be manufactured via any number of different methods of manufacture such as through the use of molds, injection molding, plastic extrusion, vacuum casting, 3d printing, suck downs, and the like.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention, are contemplated thereby, and are intended to be covered by the present invention's patent application.

What is claimed is:

1. A system for reducing grinding of teeth comprising:
an upper base member configured to fit to a user's upper teeth;
a bottom base member configured to fit to a user's bottom teeth wherein said bottom base member is separate and apart from said upper base member;
a first upper member nodule attached to said upper base member;
a first bottom member nodule attached to said bottom base member; and
a first affixing mechanism member configured to span around said first upper member nodule and said first bottom member nodule.

2. The system of claim 1 wherein when said first affixing mechanism member is positioned around said first upper member nodule and said first bottom member nodule wherein said first affixing mechanism member applies force to said first upper member nodule and said first bottom member nodule to keep said upper base member and said bottom base member pulled toward one another.

3. The system of claim 1 further comprising:
a second upper member module attached to said upper base member; and
a second bottom member nodule attached to said bottom base member.

4. The system of claim 3 wherein:
said second upper member nodule is located across from said first upper member module; and
said second bottom member nodule is located across from said first bottom member nodule.

5. The system of claim 4 further comprising:
a second affixing mechanism member configured to span around said second upper member nodule and said second bottom member nodule.

6. The system of claim 5 wherein:
said a first upper member nodule is configured with a notch for securing said first affixing mechanism member to said first upper member nodule;
said first bottom member nodule is configured with a notch for securing said first affixing mechanism member to said first bottom member nodule;
said second upper member nodule is configured with a notch for securing said second affixing mechanism member to said second upper member nodule; and
said second bottom member nodule is configured with a notch for securing said second affixing mechanism member to said second bottom member nodule.

7. The system of claim 4 wherein said affixing mechanism member is comprised of one or more of:
rubber;
nylon;
teflon; or
wire.

8. The system of claim 4 wherein said nodules are hourglass shaped.

9. A method for decreasing the symptoms associated with sleep apnea comprising:
providing a system for decreasing the symptoms associated with sleep apnea comprising:
an upper base member configured to fit to a user's upper teeth;
a bottom base member configured to fit to a user's bottom teeth wherein said bottom base member is separate and apart from said upper base member;
a first upper member nodule attached to said upper base member;
a first bottom member nodule attached to said bottom base member;
a first affixing mechanism member configured to span around said first upper member nodule and said first bottom member nodule;
placing said upper base member on said upper teeth;
placing said bottom base member on said bottom teeth; and
placing said first affixing mechanism member around said first upper member nodule and said first bottom member nodule.
placing said second affixing mechanism member around said second upper member nodule and said second bottom member nodule.

10. The method of claim 10 wherein said system for decreasing the symptoms associated with sleep apnea further comprises:
a second upper member module attached to said upper base member wherein said second upper member nodule is located across from said first upper member module;
a second bottom member nodule attached to said bottom base member wherein said second bottom member nodule is located across from said first bottom member nodule;
a second affixing mechanism member configured to span around said second upper member nodule and said second bottom member nodule;

11. The method of claim 10 further comprising the step of:
placing said second affixing mechanism member around said second upper member nodule and said second bottom member nodule.

12. A system for preventing the slackening of the jaw and preventing the constriction of the user's airway to assist with reducing the effects of sleep apnea comprising:
an upper base member configured to fit to a user's upper teeth;
a bottom base member configured to fit to a user's bottom teeth wherein said bottom base member is separate and apart from said upper base member;
a first upper member nodule attached to said upper base member;
a first bottom member nodule attached to said bottom base member; and
a first affixing mechanism member configured to span around said first upper member nodule and said first bottom member nodule.

13. The system of claim 12 further comprising:
a second upper member module attached to said upper base member; and
a second bottom member nodule attached to said bottom base member.

14. The system of claim 13 wherein:
said second upper member nodule is located across from said first upper member module; and
said second bottom member nodule is located across from said first bottom member nodule.

15. The system of claim 13 further comprising:
a second affixing mechanism member configured to span around said second upper member nodule and said second bottom member nodule.

16. The system of claim 15 wherein:
said a first upper member nodule is configured with a notch for securing said first affixing mechanism member to said first upper member nodule;
said first bottom member nodule is configured with a notch for securing said first affixing mechanism member to said first bottom member nodule;
said second upper member nodule is configured with a notch for securing said second affixing mechanism member to said second upper member nodule; and
said second bottom member nodule is configured with a notch for securing said second affixing mechanism member to said second bottom member nodule.

17. The system of claim 16 wherein said affixing mechanism member is comprised of one or more of:
rubber;
nylon;
teflon; or
wire.

18. The system of claim 15 wherein:
said first affixing mechanism member is positioned around said first upper member nodule and said first bottom member nodule wherein said first affixing mechanism member applies force to said first upper member nodule and said first bottom member nodule to keep said upper base member and said bottom base member pulled toward one another;
said second affixing mechanism member is positioned around said second upper member nodule and said second bottom member nodule wherein said second affixing mechanism member applies force to said second upper member nodule and said second bottom member nodule to keep said upper base member and said bottom base member pulled toward one another.
